(19) United States
(12) Patent Application Publication
(10) Pub. No.: US 2021/0128053 A1
(43) Pub. Date: May 6, 2021

(54) EPILEPTIC SEIZURE DETECTION WITH EEG PREPROCESSING

(71) Applicant: INTERNATIONAL BUSINESS MACHINES CORPORATION, Armonk, NY (US)

(72) Inventors: Hiroki Yanagisawa, Tokyo (JP); Toshiya Iwamori, Tokyo (JP)

(21) Appl. No.: 16/670,177

(22) Filed: Oct. 31, 2019

(51) Int. Cl. A61B 5/00; A61B 5/0476; A61B 5/04

(52) U.S. Cl. CPC A61B 5/4094; G06N 3/02; A61B 5/04012; A61B 5/0476

(57) ABSTRACT

Methods and systems for detecting seizures include generating two-dimensional frames that each include a first set of elements that store measurements from sensors and a second set of elements that store values calculated from said measurements. The two-dimensional frames are classified using a machine learning model. It is determined that a subject experienced a seizure during a measurement interval based on an output of the machine learning model. A corrective action is performed responsive to the determination that the subject experienced a seizure.

### BACKGROUND

The present invention generally relates to processing electroencephalograph (EEG) data and, more particularly, to identifying epileptic seizures from EEG data.

EEGs collect data from brain activity (e.g., “brain waves”) through electrodes attached to a subject's head. Each electrode measures a voltage at the surface of the patient's head. These voltages change in response to brain activity, as the electrical conduction of neurons in the brain causes electrical fields that can be sensed from outside the skull. Each electrode generates a time series of data. Together, these time series characterize overall brain function.

### SUMMARY

A method for detecting seizures includes generating two-dimensional frames that each include a first set of elements that store measurements from sensors and a second set of elements that store values calculated from said measurements. The two-dimensional frames are classified using a machine learning model. It is determined that a subject experienced a seizure during a measurement interval based on an output of the machine learning model. A corrective action is performed responsive to the determination that the subject experienced a seizure.

A system for detecting seizures includes a data formatter configured to generate two-dimensional frames that each include a first set of elements that store measurements from a plurality of sensors and a second set of elements that store values calculated from said measurements. A classifier is configured to classify the two-dimensional frames using a machine learning model. An interpreter is configured to determine that a subject experienced a seizure during a measurement interval based on an output of the machine learning model, and to perform a corrective action responsive to the determination that the subject experienced a seizure.

These and other features and advantages will become apparent from the following detailed description of illustrative embodiments thereof, which is to be read in connection with the accompanying drawings.

### BRIEF DESCRIPTION OF THE DRAWINGS

The following description will provide details of preferred embodiments with reference to the following figures wherein:

FIG. 1 is a diagram of a seizure detection system that includes multiple forms of data representation in an input to a neural-network based classifier in accordance with an embodiment of the present invention;

FIG. 2 is a block/flow diagram of a seizure detection method that includes multiple forms of data representation in an input to a neural-network based classifier in accordance with an embodiment of the present invention;

FIG. 3 is a diagram of a frame of sensor data that includes multiple forms of data representation, suitable for input to a neural-network based classifier in accordance with an embodiment of the present invention;

FIG. 4 is a diagram of a neural network classifier configured to accept input data that includes multiple forms of data representation in accordance with an embodiment of the present invention;

FIG. 5 is a block diagram of a seizure detection system that includes multiple forms of data representation in an input to a neural-network based classifier in accordance with an embodiment of the present invention;

FIG. 6 is a block diagram of a processing system in accordance with an embodiment of the present invention;

FIG. 7 is a diagram of a neural network classifier in accordance with an embodiment of the present invention; and

FIG. 8 is a diagram of a neural network architecture in accordance with an embodiment of the present invention.

### DETAILED DESCRIPTION

The present embodiments provide detection of epileptic seizures using electroencephalograph (EEG) data and a machine learning classifier. The EEG data is preprocessed in a manner that combines different representations of the EEG data in a manner that makes the data particularly suitable for machine learning models that are based on two-dimensional convolutional neural networks (CNNs), though it should be understood that any appropriate type of machine learning model can be used instead.

The electrodes of the EEG can be positioned on a subject's skull in a pattern referred to as a “10-20 system.” The “10” and the “20” refer to the distances between electrodes, which are either 10% or 20% of the front-to-back or right-to-left distance on the skull. The system positions, for example, twenty-one electrodes on the skull and ears of the user, with designations such as, “Fp1,” and “O2” to identify the location of the electrode relative to underlying brain structures. Thus, for example, nodes may be positioned over the pre-frontal lobe (Fp), the frontal lobe (F), the temporal lobe (T), the parietal lobe (P), the occipital lobe (0), and the central lobe (C). The designation of “A” or “M” refers to electrodes positioned just behind the ear. A number in the electrode identifier further designates the electrode's position, with odd numbers being on the left side of the skull and with even numbers being on the right side. The use of a “z” instead of a number designates an electrode that is positioned on the center line of the skull—such nodes are often used as reference points.

Thus, for example, twenty-one time series can be measured from the respective electrodes over a period of time. Rather than using the time series from the electrodes directly, the present embodiments combine multiple different representations. When processing EEG data, one representation that can be used identifies the differences between electrodes. The differences between the measured voltages can then be used to represent the brain's activity. For example, the difference between Fp1 and F7 can be plotted to show one output time series, F7 and T3 to show a second output time series, and so on. In another representation, the time series data for a single electrode can be approximated using an average potential reference or a source derivation. The present embodiments combine these two representations, measuring the differences between electrode time series and measuring single-electrode approximations, into a single two-dimensional space.

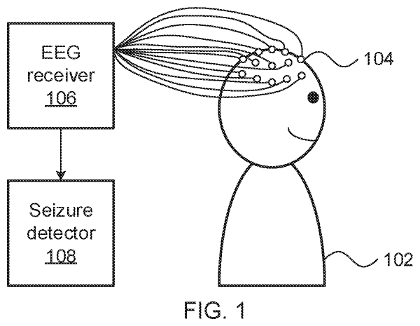

Referring now to the drawings in which like numerals represent the same or similar elements and initially to FIG. 1, a diagram of EEG electrode placement on a subject's head is shown. A set of electrodes 104 are positioned on a subject's head. Although the positioning of the electrodes 104 is described herein as being placed according to the 10-20 system, it should be understood that other positioning schemes (such as the 10-5 system) are also contemplated, for example with a denser arrangement of electrodes 104.

The electrodes 104 sense voltages at the surface of the subject's skin. These voltages are measured by the EEG receiver 106, which converts the analog voltage information into a series of measurements. The EEG receiver 106 thus outputs respective time series information for each of the electrodes 104 over the duration of the measurement. In some embodiments, the time series can be generated for a period between ten minutes and sixty minutes.

A seizure detector 108 processes the raw EEG time series to identify whether the subject 102 experienced a seizure during the measurement. As will be described in greater detail below, the seizure detector 108 preprocesses the raw EEG data to combine measurements at a particular point in time into a single frame of data, including representations of the differences between the electrodes' measurements as well as averages of neighboring electrodes.

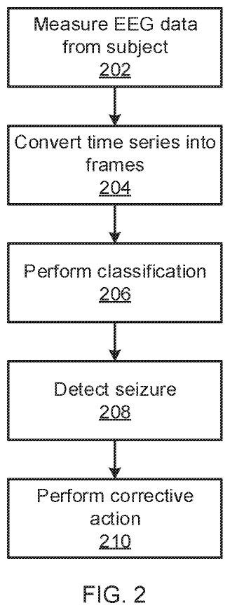

Referring now to FIG. 2, a method for detecting seizures is shown. Although the present embodiments are described in the specific context of this medical context, it should be understood that the present principles can be applied in a variety of different contexts and different kinds of sensor data. For example, the present embodiments can be applied to magnetoencephalography data as well as to non-medical types of data, such as thermal sensors placed throughout a building.

Block 202 measures a set of time series from respective sensors. As noted above, in some embodiments this time series information can include EEG data from a subject 102, collected using electrodes 104 and the EEG receiver 106. The multiple time series of the EEG data are converted into frames, as described in greater detail below, with a measurement from each time series being represented as elements of a matrix and with additional relationships between the time series, such as differences and averages, being represented as additional elements of the matrix in block 204. A frame is thus generated at each time step.

The frames are used as input to a machine learning classifier in block 206. In some embodiments, the classifier outputs a value that represents a likelihood that the subject 102 is experiencing a seizure. Block 208 uses this likelihood to determine whether the subject is experiencing a seizure, for example by comparing the output of the classifier to a threshold magnitude (in one exemplary embodiment, this threshold magnitude can be about 0.5). As noted above, the present principles are not limited to the detection of seizures, but can instead be used to detect any kind of condition in a measured subject or system.

Once a condition has been detected by block 208, block 210 performs a corrective action. In the case of detecting seizures, block 210 may include administering antiepileptic medications and prescribing other treatments to the subject. In some particular embodiments, the treatment can be administered automatically, for example with a mechanism that automatically adds an appropriate medication to an intravenous line. In this manner, the present embodiments can quickly and automatically respond to a subject's changing medical condition. In other embodiments, the detection of a seizure may not be used directly for treatment, but may be used to inform the patient of their medical condition. For example, the hiring process for pilots can include the performance of an EEG to determine whether the pilot has a condition that could make them unsuitable for the position.

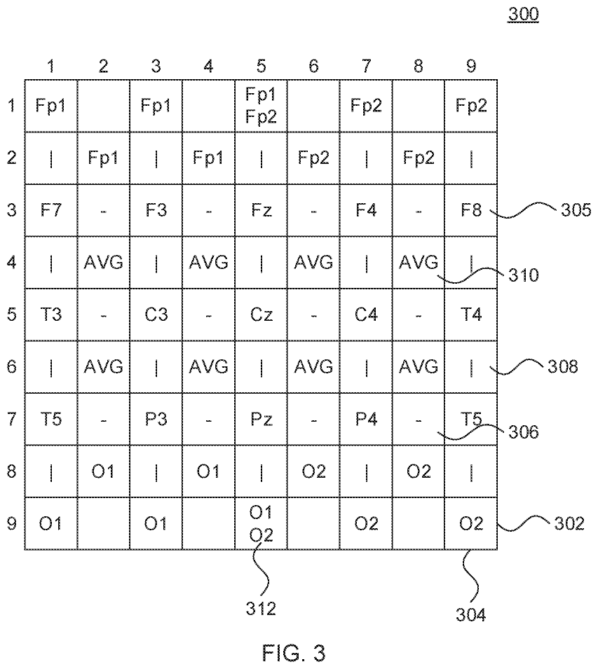

Referring now to FIG. 3, an exemplary frame format 300 is shown. As noted above, block 204 assembles the individual time series from the different electrodes 104 into frames 300. The frame is made up of a number of rows 302 and columns 304. Some elements 305 store a value of the electrode 104 corresponding to the label (e.g., the FP1 electrode) and some electrodes 104 are represented more than once. Along the center line, there are some elements 312 that are the average of the left and right side, such as the average of electrodes Fp1 and Fp2 and the average of electrodes O1 and O2.

Between the raw value elements 305, there are difference elements 306 and 308. Horizontal difference elements 306 store a magnitude of the difference between their horizontally neighboring elements 305, while vertical difference elements 308 store a magnitude of the difference between their vertically neighboring elements 305. Averaging elements 310, meanwhile, store an average of the values in the elements 305 at their corners. Thus, the indicated averaging element 310 would store the average of the values of the F4, F8, C4, and T4 electrodes.

One frame 300 can be assembled for each time value t that is registered in the time series measurements of the electrodes 104 over a time period T. Some of the values, particularly those in the first and ninth rows, may not be used as input to the classifier and may be removed. Such rows may include measurements, to provide something for the vertical difference elements 308 of adjacent rows to work with, but the elements that do not include such measurements can be left empty, or with a “zero” value. Thus, the resulting dataset can be a three-dimensional dataset having dimensions 7x9xT.

Described more generally, difference elements 306/308 are arranged in a matrix like a chessboard. The difference elements in odd columns are vertical difference elements 308, while the difference elements in even columns are horizontal difference elements 306. The size of the matrix can be made arbitrarily large to accommodate additional electrodes. Furthermore, the positioning of the electrodes in FIG. 3 can be altered, with electrode values being swapped left-to-right, with some cells being left unfilled (represented as zeros), and with some time series elements 305 being swapped for averaging elements 310 (or vice versa). In other embodiments, the frame is not limited to a chessboard arrangement. In such embodiments, there may be multiple vertically adjacent rows with a same pattern of time series and difference elements, rather than alternating every row. These variations should not be construed as limiting—any appropriate arrangement of electrodes in the frame 300 can be used.

Additionally, the frame 300 can be split down the middle, for example with a left-hand frame including columns 1-5 and with a right-hand frame including columns 5-9. In such an embodiment, the right-hand frame can be flipped to put the center column (5) at the right hand side of the frame. This allows both halves of the frame to be processed by a single filter at the same time during classification. In some embodiments, the middle three columns can be omitted, with the result being a left-hand frame that includes columns 1, 2, and 3, and a right-hand frame that includes columns 9, 8, and 7. Splitting the frame into left-hand and right-hand frames can be useful when the application does not distinguish between brain hemispheres. The frame can then be split to treat the hemispheres equally.

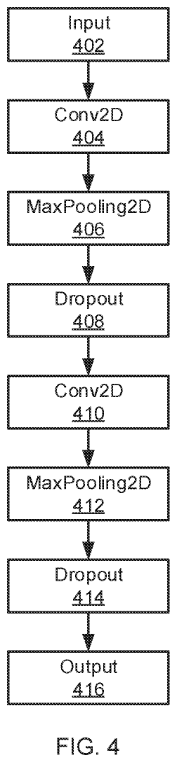

Referring now to FIG. 4, an overview of the structure of a convolutional neural network is shown, suitable for processing the EEG information. Although one particular structure is shown herein, it should be understood that any appropriate neural network structure can be used instead. The input layer 402 accepts a segment. The segment can include raw measurement data or can, in some embodiments, use a fast Fourier transform (FFT) version of the data, which transforms the time-series information into a frequency domain. The segment can furthermore include left or right hemisphere information, or both. The input 402 is processed by a first two-dimensional convolutional layer 404, a first two-dimensional max pooling layer 406, and a first dropout layer 408. The output of the first dropout layer 408 is then passed to a second two-dimensional convolutional layer 410, a second two-dimensional max pooling layer 412, and a second dropout layer 414. As described in greater detail above, the output 416 of the network can be a value between zero and one that indicates a likelihood of having detected a seizure. For example, zero can mean no likelihood of seizure, and one can mean a certainty of seizure.

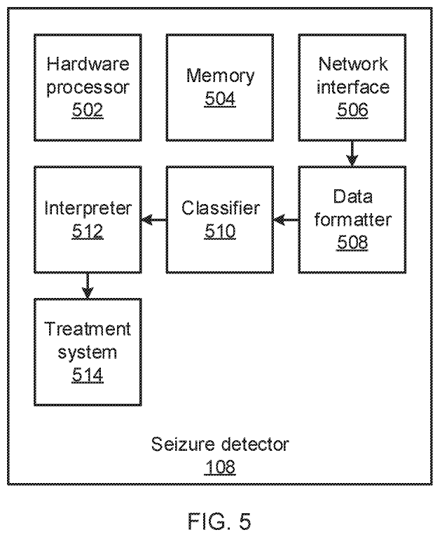

Referring now to FIG. 5, additional detail on the seizure detector 108 is shown. The detector 108 includes a hardware processor 502 and a memory 504. A network interface 506 communicates with the EEG receiver 106 by any appropriate wired or wireless network medium and protocol. The network interface 506 receives a set of time series from the EEG receiver 106, for example one time series for each electrode 104.

A data formatter 508 accepts the time series and converts them into frames 300, as described above. Classifier 510 then accepts the frames 300 and processes in the manner described in FIG. 4, generating a classifier output. Interpreter 512 determines, based on the classifier output's behavior over time, whether the subject experienced a seizure during the measured interval. If so, treatment system 514 automatically administers an appropriate treatment.

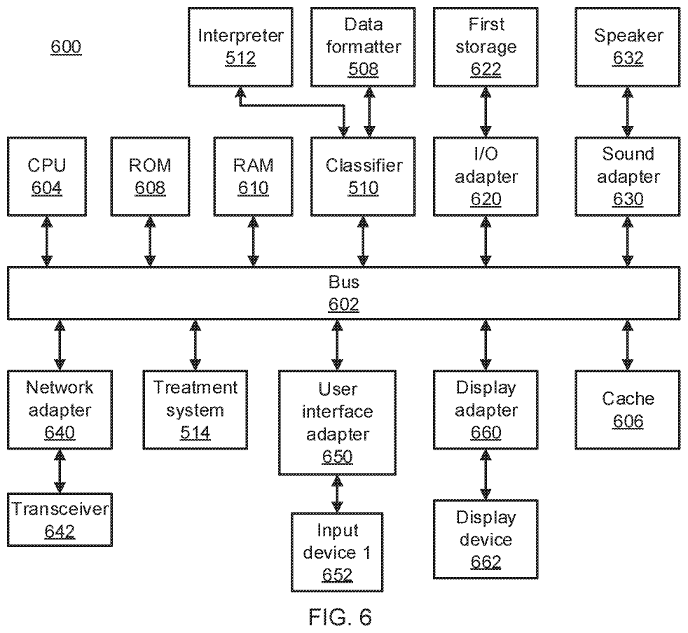

Referring now to FIG. 6, an exemplary processing system 600 is shown which may represent the seizure detector 108. The processing system 600 includes at least one processor (CPU) 604 operatively coupled to other components via a system bus 602. A cache 606, a Read Only Memory (ROM) 608, a Random Access Memory (RAM) 610, an input/output (I/O) adapter 620, a sound adapter 630, a network adapter 640, a user interface adapter 650, and a display adapter 660, are operatively coupled to the system bus 602.

A first storage device 622 is operatively coupled to system bus 602 by the I/O adapter 620. The storage device 622 can be any of a disk storage device (e.g., a magnetic or optical disk storage device), a solid state magnetic device, and so forth. The storage device 622 can be the same type of storage device or different types of storage devices.

A speaker 632 is operatively coupled to system bus 602 by the sound adapter 630. A transceiver 642 is operatively coupled to system bus 602 by network adapter 640. A display device 662 is operatively coupled to system bus 602 by display adapter 660.

A first user input device 652 is operatively coupled to system bus 602 by user interface adapter 650. The user input device 652 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present principles. The user input device 622 can be the same type of user input device or different types of user input devices. The user input device 652 is used to input and output information to and from system 600.

Of course, the processing system 600 may also include other elements (not shown), as readily contemplated by one of skill in the art, as well as omit certain elements. For example, various other input devices and/or output devices can be included in processing system 600, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 600 are readily contemplated by one of ordinary skill in the art given the teachings of the present principles provided herein.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), FPGAs, and/or PLAs.

These and other variations of a hardware processor subsystem are also contemplated in accordance with embodiments of the present invention.

An artificial neural network (ANN) is an information processing system that is inspired by biological nervous systems, such as the brain. The key element of ANNs is the structure of the information processing system, which includes a large number of highly interconnected processing elements (called “neurons”) working in parallel to solve specific problems. ANNs are furthermore trained in-use, with learning that involves adjustments to weights that exist between the neurons. An ANN is configured for a specific application, such as pattern recognition or data classification, through such a learning process.

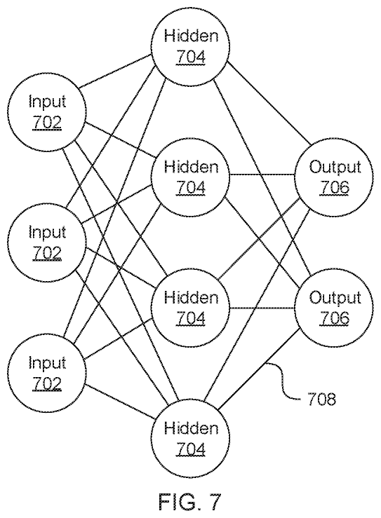

Referring now to FIG. 7, a generalized diagram of a neural network is shown. ANNs demonstrate an ability to derive meaning from complicated or imprecise data and can be used to extract patterns and detect trends that are too complex to be detected by humans or other computer-based systems. The structure of a neural network is known generally to have input neurons 702 that provide information to one or more “hidden” neurons 704. Connections 708 between the input neurons 702 and hidden neurons 704 are weighted and these weighted inputs are then processed by the hidden neurons 704 according to some function in the hidden neurons 704, with weighted connections 708 between the layers. There can be any number of layers of hidden neurons 704, and as well as neurons that perform different functions. There exist different neural network structures as well, such as convolutional neural network, maxout network, etc. Finally, a set of output neurons 706 accepts and processes weighted input from the last set of hidden neurons 704.

This represents a “feed-forward” computation, where information propagates from input neurons 702 to the output neurons 706. Upon completion of a feed-forward computation, the output is compared to a desired output available from training data. The error relative to the training data is then processed in “feed-back” computation, where the hidden neurons 704 and input neurons 702 receive information regarding the error propagating backward from the output neurons 706. Once the backward error propagation has been completed, weight updates are performed, with the weighted connections 708 being updated to account for the received error. This represents just one variety of ANN.

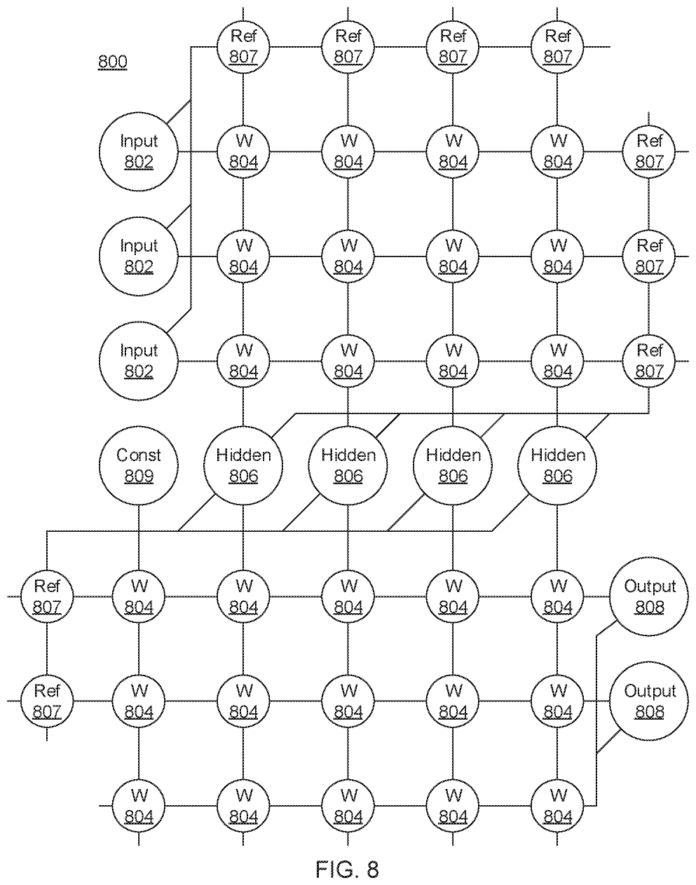

Referring now to FIG. 8, an artificial neural network (ANN) architecture 800 is shown. It should be understood that the present architecture is purely exemplary and that other architectures or types of neural network can be used instead. In particular, while a hardware embodiment of an ANN is described herein, it should be understood that neural network architectures can be implemented or simulated in software. The hardware embodiment described herein is included with the intent of illustrating general principles of neural network computation at a high level of generality and should not be construed as limiting in any way.

Furthermore, the layers of neurons described below and the weights connecting them are described in a general manner and can be replaced by any type of neural network layers with any appropriate degree or type of interconnectivity. For example, layers can include convolutional layers, pooling layers, fully connected layers, softmax layers, or any other appropriate type of neural network layer. Furthermore, layers can be added or removed as needed and the weights can be omitted for more complicated forms of interconnection.

During feed-forward operation, a set of input neurons 802 each provide an input voltage in parallel to a respective row of weights 804. In the hardware embodiment described herein, the weights 804 each have a settable resistance value, such that a current output flows from the weight 804 to a respective hidden neuron 806 to represent the weighted input. In software embodiments, the

P201901927US01 (163-2387) Page 17 of 25 weights 804 can simply be represented as coefficient values that are multiplied against the relevant neuron outputs.

Following the hardware embodiment, the current output by a given weight 804 is determined as l=v/r, where V is the input voltage from the input neuron 802 and r is the set resistance of the weight 804. The current from each weight adds column-wise and flows to a hidden neuron 806. A set of reference weights 807 have a fixed resistance and combine their outputs into a reference current that is provided to each of the hidden neurons 806. Because conductance values can only be positive numbers, some reference conductance is needed to encode both positive and negative values in the matrix. The currents produced by the weights 804 are continuously valued and positive, and therefore the reference weights 807 are used to provide a reference current, above which currents are considered to have positive values and below which currents are considered to have negative values. The use of reference weights 807 is not needed in software embodiments, where the values of outputs and weights can be precisely and directly obtained. As an alternative to using the reference weights 807, another embodiment can use separate arrays of weights 804 to capture negative values.

The hidden neurons 806 use the currents from the array of weights 804 and the reference weights 807 to perform some calculation. The hidden neurons 806 then output a voltage of their own to another array of weights 804. This array performs in the same way, with a column of weights 804 receiving a voltage from their respective hidden neuron 806 to produce a weighted current output that adds row-wise and is provided to the output neuron 808.

It should be understood that any number of these stages can be implemented, by interposing additional layers of arrays and hidden neurons 806. It should also be noted that some neurons can be constant neurons 809, which provide a constant output to the array. The constant neurons 809 can be present among the input neurons 802 and/or hidden neurons 806 and are only used during feed-forward operation.

During back propagation, the output neurons 808 provide a voltage back across the array of weights 804. The output layer compares the generated network response to training data and computes an error. The error is applied to the array as a voltage pulse, where the height and/or duration of the pulse is modulated proportional to the error value. In this example, a row of weights 804 receives a voltage from a respective output neuron 808 in parallel and converts that voltage into a current which adds column-wise to provide an input to hidden neurons 806. The hidden neurons 806 combine the weighted feedback signal with a derivative of its feed-forward calculation and stores an error value before outputting a feedback signal voltage to its respective column of weights 804. This back propagation travels through the entire network 800 until all hidden neurons 806 and the input neurons 802 have stored an error value.

During weight updates, the input neurons 802 and hidden neurons 806 apply a first weight update voltage forward and the output neurons 808 and hidden neurons 806 apply a second weight update voltage backward through the network 800. The combinations of these voltages create a state change within each weight 804, causing the weight 804 to take on a new resistance value. In this manner the weights 804 can be trained to adapt the neural network 800 to errors in its processing. It should be noted that the three modes of operation, feed forward, back propagation, and weight update, do not overlap with one another.

As noted above, the weights 804 can be implemented in software or in hardware, for example using relatively complicated weighting circuitry or using resistive cross point devices. Such resistive devices can have switching characteristics that have a non-linearity that can be used for processing data. The weights 804 can belong to a class of device called a resistive processing unit (RPU), because their non-linear characteristics are used to perform calculations in the neural network 800. The RPU devices can be implemented with resistive random access memory (RRAM), phase change memory (PCM), programmable metallization cell (PMC) memory, or any other device that has non-linear resistive switching characteristics. Such RPU devices can also be considered as memristive systems.

Having described preferred embodiments of epileptic seizure detection with EEG preprocessing (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed is:

1. A method for detecting seizures, comprising:
generating two-dimensional frames that each include a first set of elements that store measurements from a plurality of sensors and a second set of elements that store values calculated from said measurements;
classifying the two-dimensional frames using a machine learning model;
determining that a subject experienced a seizure during a measurement interval based on an output of the machine learning model; and
performing a corrective action responsive to the determination that the subject experienced a seizure.

2. The method of claim 1, wherein each two-dimensional frame includes difference elements, which store values calculated as a difference of two adjacent measurements, and average elements, which store values calculated as an average of multiple adjacent measurements.

3. The method of claim 2, wherein each two-dimensional frame includes rows that alternate between difference elements and measurements.

4. The method of claim 2, wherein each two dimensional frame includes rows that alternate between difference elements and average elements.

5. The method of claim 2, wherein each two-dimensional frame includes rows that alternate between measurements and empty or zero-value cells.

6. The method of claim 2, wherein each average cell stores an average value of four diagonally adjacent cells.

7. The method of claim 1, wherein the machine learning model is a neural network that includes a two-dimensional convolutional layer and that accepts the two-dimensional frames as input.

8. The method of claim 1, wherein generating two-dimensional frames includes generating the frames using electroencephalograph measurements.

9. The method of claim 1, wherein generating two-dimensional frames includes generating a first frame using measurements from a full set of sensors and dividing the first frame into left-hand and right-hand frames, each using measurements from a respective subset of the full set of sensors.

10. The method of claim 9, wherein generating the two-dimensional frames further includes column-wise flipping one of the left-hand frame and the right-hand frame.

11. A non-transitory computer readable storage medium comprising a computer readable program for detecting seizures, wherein the computer readable program when executed on a computer causes the computer to perform the steps of:
generating two-dimensional frames that each include a first set of elements that store measurements from a plurality of sensors and a second set of elements that store values calculated from said measurements;
classifying the two-dimensional frames using a machine learning model;
determining that a subject experienced a seizure during a measurement interval based on an output of the machine learning model; and
performing a corrective action responsive to the determination that the subject experienced a seizure.

12. A system for detecting seizures, comprising:
a data formatter configured to generate two-dimensional frames that each include a first set of elements that store measurements from a plurality of sensors and a second set of elements that store values calculated from said measurements;
a classifier configured to classify the two-dimensional frames using a machine learning model;
an interpreter configured to determine that a subject experienced a seizure during a measurement interval based on an output of the machine learning model, and to perform a corrective action responsive to the determination that the subject experienced a seizure.

13. The system of claim 12, wherein each two-dimensional frame includes difference elements, which store values calculated as a difference of two adjacent measurements, and average elements, which store values calculated as an average of multiple adjacent measurements.

14. The system of claim 13, wherein each two-dimensional frame includes rows that alternate between difference elements and measurements.

15. The system of claim 13, wherein each two dimensional frame includes rows that alternate between difference elements and average elements.

16. The system of claim 13, wherein each two-dimensional frame includes rows that alternate between measurements and empty or zero-value cells.

17. The system of claim 13, wherein each average cell stores an average value of four diagonally adjacent cells.

18. The system of claim 12, wherein the machine learning model is a neural network that includes a two-dimensional convolutional layer and that accepts the two-dimensional frames as input.

19. The system of claim 12, wherein the data formatter is further configured to generate the frames using electroencephalograph measurements.

20. The system of claim 12, wherein the data formatter is further configured to generate a first frame using measurements from a full set of sensors and dividing the first frame into left-hand and right-hand frames, each using measurements from a respective subset of the full set of sensors.
